(19) United States
(12) Patent Application Publication
(10) Pub. No.: US 2009/0202598 A1
(43) Pub. Date: Aug. 13, 2009

(54) INVERT EMULSION COMPRISING AN AQUEOUS SOLUTION OF HYDROGEN PEROXIDE AND AN INERT PHASE WITH WATER SOLUBILITY OF LESS THAN 1%

(76) Inventors: Sylvain Kravtchenko, Shanghai (CN); Claude Dubief, Le Chesnay (FR); Luc Nicolas-Morgantini, Rully (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/912,975

(22) PCT Filed: Apr. 28, 2006
(86) PCT No.: PCT/FR06/00973

§ 371 Date: Jul. 7, 2008

Related U.S. Application Data
(60) Provisional application No. 60/688,351, filed on Jun. 8, 2005.

(30) Foreign Application Priority Data
Apr. 29, 2005 (FR) 0551118

(51) Int. Cl. A61K 8/06; A61K 8/22; A61Q 5/08

(52) U.S. Cl. 424/401; 424/62

(57) ABSTRACT

The invention relates to an invert emulsion for bleaching keratin fibres and, in particular, human keratin fibres, such as hair. According to the invention, the emulsion consists of an inert phase comprising at least one non-oxygenated, non-perfluorinated liquid compound having a water solubility at 25° C. of less than 1% and an aqueous solution of hydrogen peroxide, said inert phase representing at least 20 wt.-% of the total weight of the invert emulsion and the volume-average diameter of the dispersed aqueous phase being greater than 200 nm. The invention also relates to a bleaching method which employs the inventive invert emulsion either as it is or in the form of a ready-to-use composition and to the use of said emulsion for bleaching keratin fibres. The inventive emulsion can be used to lighten keratin fibres quickly and significantly while limiting damage to said fibres and skin irritation.

The subject of the present invention is an invert emulsion for bleaching keratin fibres, and in particular human keratin fibres such as the hair.

When a person wishes to radically change hair colour, in particular when they wish to obtain a lighter colour than the original colour, it is often necessary to bleach the hair. To do this, bleaching products are used. This bleaching step is optionally combined with a step for dyeing the hair.

It is known to bleach keratin fibres, and in particular human keratin fibres such as the hair, with bleaching compositions containing one or more oxidizing agents. Among the oxidizing agents conventionally used, there may be mentioned hydrogen peroxide, compounds capable of producing hydrogen peroxide by hydrolysis, such as urea peroxide, or persalts such as perborates, percarbonates and persulphates, hydrogen peroxide and persulphates being particularly preferred.

Bleaching compositions are provided mainly in the form of anhydrous products, powders or creams, containing alkaline compounds such as amines or alkali metal silicates, and a peroxygenated reagent such as ammonium or alkali metal persulphates, perborates or percarbonates which are diluted at the time of use with an aqueous hydrogen peroxide composition.

Bleaching compositions may also result from mixing, at the time of use, an anhydrous powder containing the peroxygenated reagent with an aqueous composition containing the alkaline compounds and another aqueous composition containing hydrogen peroxide.

To obtain a product for bleaching keratin fibres which is more effective in terms of lightening and/or speed, it is in theory possible to increase the hydrogen peroxide concentration in the aqueous hydrogen peroxide composition. However, a high hydrogen peroxide concentration can cause degradation of the keratin fibres and possibly skin irritation. Conventionally, in conventional bleaching compositions, the hydrogen peroxide concentration is therefore limited to 12% by weight, or even to 6% by weight.

Moreover, patent application EP 0,193,471 proposes bleaching the hair with an anhydrous hydrogen peroxide solution in an organic solvent.

There has also been proposed, in patent application WO 03/011216, a composition for bleaching the hair which is provided in the form of a direct or inverse emulsion containing an aqueous hydrogen peroxide solution and an organic phase comprising at least one perfluorinated compound.

However, the results obtained in terms of lightening are not yet satisfactory.

The aim of the present invention is to provide novel lightening products which are more effective than the lightening products known in the prior art while limiting degradation of keratin fibres and skin irritation.

This aim is achieved with the present invention whose subject is an invert emulsion for bleaching keratin fibres, comprising an inert phase comprising at least one nonoxygenated and nonperfluorinated liquid compound having a water-solubility at 25° C. of less than 1% and an aqueous hydrogen peroxide solution, the inert phase representing at least 20% by weight of the total weight of the invert emulsion and the mean diameter by volume of the dispersed aqueous phase being greater than 200 nm.

The invert emulsion in accordance with the invention makes it possible to rapidly obtain substantial lightening of keratin fibres while limiting the degradation of the keratin fibres and skin irritation.

The subject of the present invention is also a ready-to-use bleaching composition resulting from mixing the invert emulsion in accordance with the invention with one or more compositions each comprising at least one compound chosen from an alkaline agent, a persalt, a direct dye and an oxidation dye precursor.

The subject of the present invention is also a method for bleaching keratin fibres using the invert emulsion or the ready-to-use bleaching composition in accordance with the invention, and a kit for carrying out this method.

Another subject of the invention is the use of the invert emulsion or of the ready-to-use bleaching composition in accordance with the invention for bleaching keratin fibres.

In the context of the invention, invert emulsion is understood to mean an emulsion containing a dispersed aqueous phase.

In the context of the present invention, inert phase is understood to mean a phase which is chemically inert towards hydrogen peroxide. In the context of the invention, a phase is inert if the degradation of hydrogen peroxide in the presence of this phase is less than 25% after 15 hours at 100° C.

In the context of the present invention, a nonoxygenated and nonperfluorinated liquid compound is understood to mean any nonoxygenated or nonperfluorinated organic or inorganic derivative capable of running and having a viscosity at 25° C. of less than 5000 Poises at a shear rate of 1 s⁻¹. This viscosity may be determined with the aid of a RHEOMAT 180 type viscometer.

According to a particular embodiment of the invention, the nonoxygenated and nonperfluorinated liquid compound(s) are chosen from polyalphaolefins, polybutenes and polyisobutenes, mineral oils, paraffin oils, polystyrene and polyisoprene block copolymers such as liquid Kratons, and mixtures thereof.

According to a particular embodiment, the inert phase represents between 20 and 95% by weight, and preferably between 30 and 90% by weight of the total weight of the invert emulsion.

The mean diameter by volume of the dispersed aqueous phase is preferably between 200 nm and 100 μm. It may be determined with the aid of a laser granulometer such as the Master Sizer 2000 granulometer from the company MALVERN.

The invert emulsion in accordance with the invention generally has a hydrogen peroxide concentration of between 1 and 20% by weight, preferably between 2 and 12% by weight of the total weight of the invert emulsion.

According to a particular embodiment, the invert emulsion of the invention comprises at least one compound chosen from anionic, nonionic, cationic and amphoteric surfactants, for example those having one or more linear or branched C₆-C₂₂ alkylated chains and/or one or more perfluorinated chains, and anionic, nonionic, amphoteric and cationic derivatives of polyisobutylene succinic anhydrides as described in the book by J. M. Carey et al., Improved High Internal Phase Emulsions using Alkenyl Succinic Anhydride Based Emulsifiers, World Surfactants Congress, 5th, Firenze, Italy, May-June, 2000, 905-910, amphiphilic polymers such as water-soluble or water-dispersible amphiphilic block copolymers, oxyethylenated or nonoxyethylenated, telechelic or graft associative polymers bearing on their main chain C₆-C₂₂ alkyl and/or aryl groups, it being possible for these polymers to have a cationic, anionic, amphoteric or nonionic charge, and thickening polymers such as crosslinked acrylic polymers, natural or modified polysaccharides.

The compound(s) chosen from derivatives of polyisobutylene succinic anhydrides, amphiphilic polymers and thickening polymers are each present in a quantity of between 0.1 and 30% by weight, and preferably between 0.2 and 15% by weight of the total weight of the invert emulsion.

By way of surfactants useful in the invention, there may be mentioned nonsilicone anionic surfactants, in particular the salts of the following compounds: alkyl sulphates, alkyl ether sulphates, alkylamidoether sulphates, alkyl aryl polyether sulphates, monoglyceride sulphates; alkyl sulphonates, alkyl phosphates, alkylamide sulphonates, alkyl aryl sulphonates, α-olefin sulphonates, paraffin sulphonates; alkyl sulphosuccinates, alkyl ether sulphosuccinates, alkylamide sulphosuccinates; alkyl sulphosuccinamates; alkyl sulphoacetates; alkyl ether phosphates; acyl sarcosinates; acyl isethionates and N-acyltaurates, the alkyl or acyl radical of all these various compounds preferably containing 12 to 20 carbon atoms, and the aryl radical preferably denoting a phenyl or benzyl group. The salts of these compounds are for example alkali metal, in particular sodium, salts, ammonium salts, amine salts, salts of amino alcohols or magnesium salts. Among the anionic surfactants, there may also be mentioned the salts of fatty acids such as the salts of oleic, ricinoleic, palmitic and stearic acids, copra oil or hydrogenated copra oil acids; acyl lactylates in which the acyl radical contains from 8 to 20 carbon atoms. It is also possible to use weakly anionic surfactants such as alkyl D-galactoside uronic acids and their salts as well as polyoxyalkylenated ether carboxylic acids and their salts, in particular those containing from 2 to 50 ethylene oxide groups, and mixtures thereof. The anionic surfactants of the polyoxyalkylenated ether carboxylic salt or acid type are in particular those which correspond to the following formula (1)

R₁OC₂H₄ₙOCH₂COOA (1)

in which:

R₁ represents an alkyl or alkylaryl group, and n is an integer or a decimal number (mean value) which may vary from 2 to 24 and preferably from 3 to 10, the alkyl radical having between 6 and 20 carbon atoms approximately, and aryl preferably denoting phenyl, A represents a hydrogen atom, an ammonium, a sodium, a potassium, lithium or magnesium ion or a monoethanol-amine or triethanolamine residue.

It is also possible to use mixtures of compounds of formula (1), in particular mixtures in which the R₁ groups are different.

Among all these anionic surfactants, the use of the salts of alkyl sulphates and of alkyl ether sulphates, and mixtures thereof, is preferred.

As nonsilicone amphoteric surfactants, it is possible to use aliphatic secondary or tertiary amine derivatives in which the aliphatic radical is a linear or branched chain containing from 8 to 22 carbon atoms and containing at least one water-solubilizing anionic group, for example carboxylate, sulphonate, sulphate, phosphate or phosphonate; there may also be mentioned (C₈-C₂₀)alkylbetaines, sulphobetaines, (C₈-C₂₀)alkylamido(C₁-C₆)alkylbetaines or (C₈-C₂₀)alkylamido(C₁-C₆)-alkylsulphobetaines.

Among the amine derivatives, there may be mentioned the products marketed under the name MIRANOL, as described in Patents U.S. Pat. No. 2,528,378 and U.S. Pat. No. 2,781,354 and having the structures:

R₂—CONHCH₂CH₂ —N(R₃)(R₄)(CH₂COO⁻) (2)

in which:

R₂ denotes an alkyl radical derived from a R₂—COOH acid present in hydrolysed copra oil, a heptyl, nonyl or undecyl radical,

R₃ denotes a beta-hydroxyethyl group; and

R₄ a carboxymethyl group; and

R₅—CONHCH₂CH₂—N(B)(C) (3)

in which:

B represents —CH₂CH₂OX′, C represents —(CH₂)_{z}—Y′, with z=1 or 2,

X′ denotes the group —CH₂CH₂—COOH or a hydrogen atom,

Y′ denotes —COOH or the radical —CH₂—CHOH—SO₃H,

R₅ denotes an alkyl radical of an acid R₅—COOH present in copra oil or in hydrolysed linseed oil, an alkyl, in particular a C₇, C₉, C₁₁ or C₁₃ alkyl, radical, a C₁₇ alkyl radical and its isoform, an unsaturated C₁₇ radical.

These compounds are classified in the CTFA dictionary, 5th edition, 1993, under the names Disodium Cocoamphodiacetate, Disodium Lauroamphodiacetate, Disodium Caprylamphodiacetate, Disodium Capryloamphodiacetate, Disodium Cocoamphodipropionate, Disodium Lauroamphodipropionate, Disodium Caprylamphodipropionate, Disodium Caprylamphodipropionate, Lauroamphodipropionic acid, Cocoamphodipropionic acid.

By way of example, there may be mentioned the cocoamphodiacetate marketed under the trade name MIRANOL C2M concentré by the company RHONE POULENC.

By way of nonsilicone nonionic surfactant(s), there may be mentioned the nonionic surfactants which are well known per se (see in particular in this regard “Handbook of Surfactants” by M. R. PORTER, Blackie & Son publishers (Glasgow and London), 1991, pp. 116-178). They may be chosen in particular from (nonlimiting list) alcohols, alpha-diols, alkylphenols and polyethoxylated, polypropoxylated or polyglycerolated fatty acids having a fatty chain containing for example from 8 to 18 carbon atoms, it being possible for the number of ethylene oxide or propylene oxide groups to range in particular from 2 to 50 and it being possible for the number of glycerol groups to range in particular from 2 to 30. It is also possible to mention copolymers of ethylene and propylene oxide, condensates of ethylene and propylene oxide with fatty alcohols; polyethoxylated fatty amides preferably having from 2 to 30 moles of ethylene oxide, polyglycerolated fatty amides containing on average 1 to 5 glycerol groups and in particular 1.5 to 4; polyethoxylated fatty amines preferably having from 2 to 30 moles of ethylene oxide; oxyethylenated fatty acid esters of sorbitan having from 2 to 30 moles of ethylene oxide; fatty acid esters of sucrose, fatty acid esters of polyethylene glycol, alkyl polyglycosides, N-alkylglucamine derivatives, amine oxides such as (C₁₀-C₁₄)alkylamine oxides or N-acylaminopropylmorpholine oxides. According to one embodiment of the invention, the alkyl polyglycosides constitute nonionic surfactants useful in the context of the present invention.

As nonsilicone cationic surfactants, it is possible to use surfactants well known per se, such as the salts of optionally polyoxyalkylenated primary, secondary or tertiary fatty amines, quaternary ammonium salts, and mixtures thereof.

As quaternary ammonium salts, there may be mentioned in particular:
those which have the following formula:

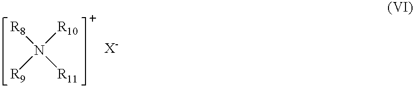

in which the radicals R₈ to R₁₁, which may be identical or different, represent a linear or branched aliphatic radical containing from 1 to 30 carbon atoms, or an aromatic radical such as aryl or alkylaryl. The aliphatic radicals may contain heteroatoms such as in particular oxygen, nitrogen, sulphur and halogens. The aliphatic radicals are for example chosen from alkyl, alkoxy, polyoxy(C₂-C₆)alkylene, alkylamide, (C₁₂-C₂₂)alkylamido(C₂-C₆)alkyl, (C₁₂-C₂₂) alkyl acetate, hydroxyalkyl radicals, concerning about 1 to 30 carbon atoms; X is an anion chosen from the group comprising halides, phosphates, acetates, lactates, (C₂-C₆)alkyl sulphates, alkyl- or alkylaryl sulphonates;
quaternary ammonium salts of imidazoline, such as for example those of the following formula:

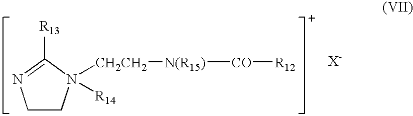

in which R₁₂ represents an alkenyl or alkyl radical containing from 8 to 30 carbon atoms, for example derivatives of tallow fatty acids, R₁₃ represents a hydrogen atom, a C₁-C₄ alkyl radical or an alkenyl or alkyl radical containing from 8 to 30 carbon atoms, R₁₄ represents a C₁-C₄ alkyl radical, R₁₅ represents a hydrogen atom, a C₁-C₄ alkyl radical, X⁻ is an anion chosen from the group comprising halides, phosphates, acetates, lactates, alkyl sulphates, alkyl- or alkylaryl sulphonates. Preferably, R₁₂ and R₁₃ denote a mixture of alkenyl or alkyl radicals containing from 12 to 21 carbon atoms, for example derivatives of tallow fatty acids, R₁₄ denotes a methyl radical, R₁₅ denotes a hydrogen atom. Such a product is for example marketed under the name REWOQUAT® W 75 by the company REWO;
quaternary diammonium salts of formula (VIII):

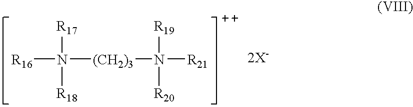

in which R₁₆ denotes an aliphatic radical containing about 16 to 30 carbon atoms, R₁₇, R₁₈, R₁₉, R₂₀ and R₂₁, which are identical or different, are chosen from hydrogen or an alkyl radical containing from 1 to 4 carbon atoms, and X is an anion chosen from the group comprising halides, acetates, phosphates, nitrates and methyl sulphates. Such quaternary diammonium salts comprise in particular propanetallowdiammonium dichloride;
quaternary ammonium salts containing at least one ester functional group, such as those of the following formula (IX):

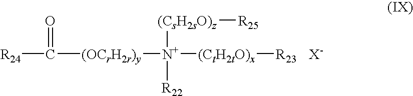

in which:

R₂₂ is chosen from C₁-C₆ alkyl radicals and C₁-C₆ hydroxyalkyl or dihydroxyalkyl radicals;

R₂₃ is chosen from:

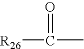

the radical
saturated or unsaturated, linear or branched C₁-C₂₂ hydrocarbon radicals R₂₇,
a hydrogen atom,

R₂₅ is chosen from:

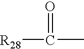

the radical
saturated or unsaturated, linear or branched C₁-C₆ hydrocarbon radicals R₂₉
a hydrogen atom,

R₂₄, R₂₆, R₂₈, which are identical or different, are chosen from saturated or unsaturated, linear or branched C₇-C₁₂ hydrocarbon radicals;

r, s and t, which are identical or different, are integers equal to 2 to 6;

y is an integer equal to 1 to 10;

x and z, which are identical or different, are integers equal to 0 to 10;

X⁻ is a simple or complex, organic or inorganic anion; provided that the sum x+y+z equals 1 to 15, when x equals 0 then R₂₃ denotes R₂₇ and that when z equals 0 then R₂₅ denotes R₂₉.

The alkyl radicals R₂₂ may be linear or branched and more particularly linear.

Preferably, R₂₂ denotes a methyl, ethyl, hydroxyethyl or dihydroxypropyl radical, and more particularly a methyl or ethyl radical.

Advantageously, the sum x+y+z equals 1 to 10.

When R₂₃ is a hydrocarbon radical R₂₇, it may be long and may have from 12 to 22 carbon atoms, or it may be short and may have from 1 to 3 carbon atoms.

When R₂₅ is a hydrocarbon radical R₂₉, it preferably has from 1 to 3 carbon atoms.

Advantageously, R₂₄, R₂₆ and R₂₈, which are identical or different, are chosen from saturated or unsaturated, linear or branched C₁₁-C₂₁ hydrocarbon radicals, and more particularly from saturated or unsaturated, linear or branched C₁₁-C₂₁ alkyl and alkenyl radicals.

Preferably, x and z, which are identical or different, equal 0 or 1.

Advantageously, y is equal to 1.

Preferably, r, s and t, which are identical or different, equal 2 or 3, and more particularly still are equal to 2.

The anion is preferably a halide (chloride, bromide or iodide) or an alkyl sulphate, more particularly methyl sulphate. It is possible, nevertheless, to use methanesulphonate, phosphate, nitrate, tosylate, an anion derived from an organic acid such as acetate or lactate or any other anion compatible with ammonium with an ester functional group.

The anion X is more particularly still chloride or methyl sulphate.

There are used more particularly in the composition according to the invention the ammonium salts of formula (IX) in which:

R₂₂ denotes a methyl or ethyl radical,

x and y are equal to 1;

z is equal to 0 or 1;

r, s and t are equal to 2;

R₂₃ is chosen from:

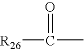

the radical
methyl, ethyl or C₁₄-C₂₂ hydrocarbon radicals,
a hydrogen atom,

R₂₅ is chosen from:

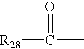

the radical
a hydrogen atom;

R₂₄, R₂₆ and R₂₈, which are identical or different, are chosen from saturated or unsaturated, linear or branched C₁₃-C₁₇ hydrocarbon radicals, and preferably from saturated or unsaturated, linear or branched C₁₃-C₁₇ alkyl and alkenyl radicals.

Advantageously, the hydrocarbon radicals are linear.

There may be mentioned, for example, the compounds of formula (IX) such as salts (chloride or methyl sulphate in particular) of diacyloxyethyldimethylammonium, diacyloxyethylhydroxyethylmethylammonium, monoacyloxyethyldihydroxyethylmethylammonium, triacyloxyethylmethylammonium, monoacyloxyethylhydroxyethyldimethylammonium and mixtures thereof. The acyl radicals preferably have from 14 to 18 carbon atoms and are derived more particularly from a vegetable oil such as palm or sunflower oil. When the compound contains several acyl radicals, the latter may be identical or different.

These products are obtained, for example, by direct esterification of triethanolamine, triisopropanolamine, alkyldiethanolamine or alkyldiisopropanolamine which are optionally oxyalkylenated on fatty acids or on mixtures of fatty acids of plant or animal origin, or by transesterification of their methyl esters. This esterification is followed by quaternization with the aid of an alkylating agent such as an alkyl (preferably methyl or ethyl) halide, a dialkyl (preferably methyl or ethyl) sulphate, methyl methanesulphonate, methyl para-toluenesulphonate, glycol or glycerol chlorohydrin.

Such compounds are for example marketed under the names DEHYQUART® by the company HENKEL, STEPANQUAT® by the company STEPAN, NOXAMIUM® by the company CECA, REWOQUAT® WE 18 by the company REWO-WITCO.

As mixture of ammonium salts, it is possible to use, for example, the mixture containing from 15 to 30% by weight of acyloxyethyldihydroxyethylmethylammonium methyl sulphate, 45 to 60% of diacyloxyethylhydroxyethylmethylammonium methyl sulphate and 15 to 30% of triacyloxyethylmethylammonium methyl sulphate, the acyl radicals having from 14 to 18 carbon atoms and being derived from optionally partially hydrogenated palm oil.

It is also possible to use ammonium salts containing at least one ester functional group which are described in patents U.S. Pat. No. 4,874,554 and U.S. Pat. No. 4,137,180.

Among the quaternary ammonium salts of formula (VI), preference is given, on the one hand, to tetraalkylammonium chlorides such as, for example, dialkyldimethylammonium or alkyltrimethylammonium chlorides in which the alkyl radical contains about 12 to 22 carbon atoms, in particular behenyltrimethylammonium, distearyldimethylammonium, cetyltrimethylammonium or benzyldimethylstearylammonium chlorides, or alternatively, on the other hand, palmitylamidopropyltrimethylammonium chloride or stearamidopropyldimethyl-(myristyl acetate)-ammonium chloride marketed under the name CERAPHYL® 70 by the company VAN DYK.

The particularly preferred cationic surfactants are chosen from quaternary ammonium salts, and in particular from behenyltrimethylammonium chloride and palmitylamidopropyltrimethylammonium chloride.

As surfactants which can be used in the present invention, there may also be particularly mentioned the silicone surfactants described in particular in Patent FR 2 818 902. The silicone surfactants which can be used in the present invention are those well known to persons skilled in the art. They may be nonionic, anionic, cationic or amphoteric. They may be water-soluble, spontaneously water-dispersible or non-water-soluble. Preferably, they are water-soluble or spontaneously water-dispersible,

The silicone surfactants are, for example, chosen from compounds of the following general formulae:

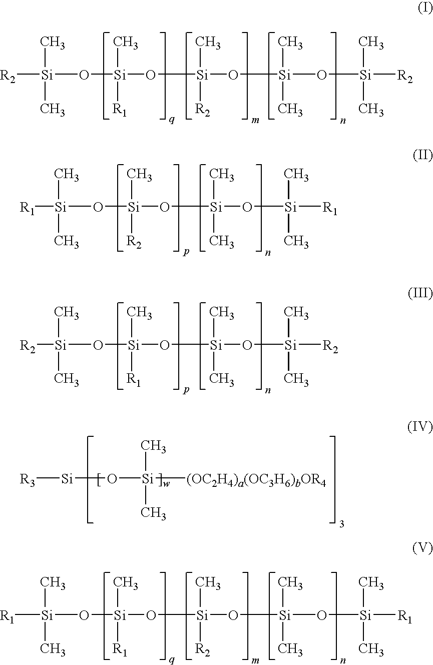

in which formulae:

R₁, which are identical or different, represent a linear or branched C₁-C₃₀ alkyl group or a phenyl group; R₂, which are identical or different, represent —C_{c}H_{2c}—O—(C₂H₄O)ₐ—(C₃H₆O)_{b}—R₅ or —C_{c}H₂C—O—(C₄H₆O)ₐ—R₅;

R₃ and R₄, which are identical or different, each denote a linear or branched C₁-C₁₂ alkyl group, and preferably a methyl group;

R₅, which are identical or different, are chosen from a hydrogen atom, a linear or branched alkyl group containing from 1 to 12 carbon atoms, a linear or branched alkoxy group containing from 1 to 6 carbon atoms, a linear or branched acyl group containing from 2 to 12 carbon atoms, a hydroxyl group, a group —SO₃M, —OCOR₆, a C₁-C₆ aminoalkoxy group optionally substituted on the amine, a C₂-C₆ aminoacyl group optionally substituted on the amine, a group —NHCH₂CH₂COOM, —N(CH₂CH₂COOM)₂, a C₁-C₁₂ aminoalkyl group optionally substituted on the amine and on the alkyl chain, a C₁-C₃₀ carboxyacyl group, a phosphono group optionally substituted with one or two substituted C₁-C₁₂ aminoalkyl groups, a group —CO(CH₂)_{d}COOM, —OCOCHR₇(CH₂)_{d}COOM, —NHCO(CH₂)_{d}OH, —NH₃Y;

M, which are identical or different, denote a hydrogen atom, Na, K, Li, NH₄ or an organic amine;

R₆ denotes a linear or branched C₁-C₃₀ alkyl group,

R₇ denotes a hydrogen atom or a group SO₃M;

d varies from 1 to 10;

m varies from 0 to 20;

n varies from 0 to 500;

p varies from 1 to 50;

q varies from 0 to 20;

a varies from 0 to 50;

b varies from 0 to 50;

a+b is greater than or equal to 1;

c varies from 0 to 4;

w varies from 1 to 100;

Y represents a monovalent inorganic or organic anion such as a halide (chloride, bromide), a sulphate or a carboxylate (acetate, lactate, citrate).

Preferably, use is made of the silicone surfactants corresponding to the general formulae (IV) or (VII) as defined above, and more particularly those corresponding to the formulae (IV) or (VII) in which at least one, and preferably all, of the following conditions are met:

c is equal to 2 or 3;

R₁ denotes a methyl group;

R₅ represents a hydrogen atom, a methyl group or an acetyl group and preferably a hydrogen atom;

a varies from 1 to 25 and more particularly from 2 to 25;

b varies from 0 to 25, preferably from 10 to 20;

n varies from 0 to 100;

p varies from 1 to 20.

The most particularly preferred silicone surfactants are, for example, those sold under the trade names FLUID DC 193 and DC 5225C by the company DOW CORNING, SILWET® L 77 by the company OSI and MAZIL® 756 by the company MAZER PPG, the LAURYL PEG/PPG-18/18 METHICONE (and) POLOXAMER 407 (and) DODECENE mixture marketed by Dow Corning under the name DC 5200.

The most particularly preferred silicone surfactants are, for example, those sold under the trade names FLUID DC 193 and DC 5225C by the company DOW CORNING, SILWET® L 77 by the company OSI and MAZIL® 756 by the company MAZER PPG, the LAURYL PEG/PPG-18/18 METHICONE (and) POLOXAMER 407 (and) DODECENE mixture marketed by Dow Corning under the name DC 5200.

The surfactants having an HLB (Hydrophilic Lipophilic Balance) of less than 10 are preferred. In particular, the nonionic surfactants used in the compositions of the invention preferably have an HLB ranging from 1.5 to 10, and better still from 1.5 to 7The HLB or hydrophilic-lipophilic balance of the nonionic surfactant(s) used according to the invention is the HLB according to GRIFFIN defined in the publication J. Soc. Cosm. Chem. 1954 (Volume 5), pages 249-256.

According to a preferred embodiment, the surfactant(s) present in the emulsion are chosen from silicone surfactants.

The surfactants may each be present in the invert emulsion according to the invention in a quantity of between 0.1 and 30% by weight, and preferably between 0.2 and 15% by weight of the total weight of the invert emulsion.

The inert phase of the emulsion may contain a specific gelling agent/structuring agent such as the silicone polyamides PSPA (DP100 and DPI5) marketed by Dow Chemical, the organic KSGs polylauryldimethylsiloxane and the silicone KSGs marketed by Shinetsu, dextrin palmitate and inulin stearate known under the name of the Rheopearl range from Chiba Flour Milling); acrylates containing an alkyl chain marketed by Landec, ethylene octene copolymers marketed by Dupont de Nemours, dibutyl lauryl glutamide marketed by Ajinomoto, disorbene marketed by Roquette, styrene acrylate copolymers Versagel M5960 and 5670 marketed by Penreco, diblock and triblock kratons marketed by Kraton Polymers, hydroxystearic acid, jojoba waxes, pyrogenic silicas marketed by Degussa, silicone waxes marketed by Waker, polyamide Uniclear marketed by Arizona Chemical, bentone.

The inert phase may comprise other oils such as nonoxygenated or nonperfluorinated liquid compounds.

The aqueous phase of the invert emulsion in accordance with the invention has a pH of less than 5. The pH may be adjusted to the desired value by means of acidic or alkaline agents customarily used in cosmetics or with the aid of conventional buffer systems.

Among the acidic agents, there may be mentioned, by way of example, inorganic or organic acids such as hydrochloric acid, orthophosphoric acid, sulphuric acid, carboxylic acids such as acetic acid, tartaric acid, citric acid, lactic acid, and sulphonic acids.

Among the alkaline agents, there may be mentioned, by way of example, ammonium hydroxide, alkali metal carbonates, alkanolamines such as di- and triethanolamines and their derivatives, sodium or potassium hydroxides and the compounds of the following formula (X):

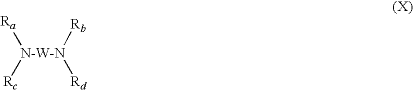

in which W is a propylene residue optionally substituted with a hydroxyl group or a C₁-C₄ alkyl radical; Rₐ, R_{b}, R_{c} and R_{d}, which may be identical or different, represent a hydrogen atom, a C₁-C₄ alkyl radical or a C₁-C₄ hydroxyalkyl radical.

To bleach keratin fibres, the invert emulsion in accordance with the invention may be applied as it is to the keratin fibres. It may also be mixed at the time of use with one or more compositions in which at least one alkaline agent and/or at least one persalt and/or at least one direct dye and/or at least one oxidation dye precursor are distributed in order to give a ready-to-use bleaching composition.

In the case where the ready-to-use bleaching composition comprises at least one direct dye and/or at least one oxidation dye precursor, lightening dyeing is carried out, that is to say that bleaching and dyeing of the keratin fibres are simultaneously obtained.

The alkaline agent(s) may be chosen from organic amines, ammonia and silicates.

When the ready-to-use bleaching composition in accordance with the invention comprises one or more alkaline agents, they are generally present in a quantity of between 0.01 and 40% by weight, and preferably between 0.1 and 30% by weight of the total weight of the ready-to-use composition.

The persalt(s) may be chosen from ammonium or alkali metal perborates, percarbonates and persulphates.

When the ready-to-use bleaching composition in accordance with the invention comprises one or more persalts, they are generally present in a quantity of between 10 and 70% by weight, and preferably between 20 and 60% by weight of the total weight of the ready-to-use composition.

The direct dye(s) may be chosen from the direct dyes conventionally used in direct dyeing. By way of examples, these direct dyes are chosen from nitro dyes of the benzene series, azo direct dyes, methine direct dyes, quinone direct dyes, azine direct dyes, triarylmethane direct dyes, indoamine direct dyes, natural direct dyes. These direct dyes may be of a nonionic, anionic or cationic nature.

Among the benzene direct dyes, there may be mentioned 1,4-diamino-2-nitrobenzene, 1-amino-2-nitro-4-(β-hydroxyethylamino)benzene, 1-amino-2-nitro-4-bis(β-hydroxyethyl)aminobenzene, 1,4-bis(β-hydroxyethylamino)-2-nitrobenzene, 1-β-hydroxyethylamino-2-nitro-4-bis(β-hydroxyethylamino)benzene, 1-β-hydroxyethylamino-2-nitro-4-aminobenzene, 1-β-hydroxyethylamino-2-nitro-4-(ethyl) (β-hydroxyethyl)aminobenzene, 1-amino-3-methyl-4-β-hydroxyethylamino-6-nitrobenzene, 1-amino-2-nitro-4-β-hydroxyethylamino-5-chlorobenzene, 1,2-diamino-4-nitrobenzene, 1-amino-2-β-hydroxyethylamino-5-nitrobenzene, 1,2-bis(β-hydroxyethylamino)-4-nitrobenzene, 1-amino-2-[tris(hydroxymethyl)methylamino]-5-nitrobenzene, 1-hydroxy-2-amino-5-nitrobenzene, 1-hydroxy-2-amino-4-nitrobenzene, 1-hydroxy-3-nitro-4-aminobenzene, 1-hydroxy-2-amino-4,6-dinitrobenzene, 1-β-hydroxyethyloxy-2-β-hydroxyethylamino-5-nitrobenzene, 1-methoxy-2-β-hydroxyethylamino-5-nitrobenzene, 1-β-hydroxyethyloxy-3-methylamino-4-nitrobenzene, 1-β,γ-dihydroxypropyloxy-3-methylamino-4-nitrobenzene, 1-β-hydroxyethylamino-4-β,γ-dihydroxypropyloxy-2-nitrobenzene, 1-β,γ-dihydroxypropylamino-4-trifluoromethyl-2-nitrobenzene, 1-β-hydroxyethylamino-4-trifluoromethyl-2-nitrobenzene, 1-β-hydroxyethylamino-3-methyl-2-nitrobenzene, 1-β-aminoethylamino-5-methoxy-2-nitrobenzene, 1-hydroxy-2-chloro-6-ethylamino-4-nitrobenzene, 1-hydroxy-2-chloro-6-amino-4-nitrobenzene, 1-hydroxy-6-[bis(β-hydroxyethyl)amino]-3-nitrobenzene, 1β-hydroxyethylamino-2-nitrobenzene, 1-hydroxy-4-β-hydroxyethylamino-3-nitrobenzene.

Among the azo direct dyes, there may be mentioned the cationic azo dyes described in patent applications WO 95/15144, WO 95/01772, ES 0,714,954, FR 2,822,696, FR 2,825,702, FR 2,825,625, FR 2,822,698, FR 2,822,693, FR 2,822,694, FR 2,829,926, FR 2,807,650, WO 02/078660, WO 02/100834, WO 02/100369 and FR 2,844,269 whose content forms an integral part of the invention.

Among these compounds, there may be mentioned most particularly 1,3-dimethyl-2-[[4-(dimethylamino)phenyl]-azo]-1H-imidazolium chloride, 1,3-dimethyl-2-[(4-aminophenyl)azo]-1H-imidazolium chloride, 1-methyl-4-[(methylphenylhydrazono)methyl]pyridinium methylsulphate.

The following dyes, which are described in COLOUR INDEX INTERNATIONAL 3^{rd} edition, may also be mentioned among the azo direct dyes: Disperse Red 17, Acid Yellow 9, Acid Black 1, Basic Red 22, Basic Red 76, Basic Yellow 57, Basic Brown 16, Acid Yellow 36, Acid Orange 7, Acid Red 33, Acid Red 35, Basic Brown 17, Acid Yellow 23, Acid Orange 24, Disperse Black 9.

There may also be mentioned 1-(4′-aminodiphenylazo)-2-methyl-4-bis(β-hydroxyethyl)aminobenzene and 4-hydroxy-3-(2-methoxyphenylazo)-1-naphthalenesulphonic acid.

The following dyes may be mentioned among the quinone direct dyes: Disperse Red 15, Solvent Violet 13, Acid Violet 43, Disperse Violet 1, Disperse Violet 4, Disperse Blue 1, Disperse Violet S, Disperse Blue 3, Disperse Red 11, Acid Blue 62, Disperse Blue 7, Basic Blue 22, Disperse Violet 15, Basic Blue 99, and the following compounds: 1-N-methylmorpholiniumpropylamino-4-hydroxyanthraquinone, 1-aminopropylamino-4-methylaminoanthraquinone, 1-aminopropylaminoanthraquinone, 5-β-hydroxyethyl-1,4-diaminoanthraquinone, 2-aminoethylaminoanthraquinone, 1,4bis(β,?-dihydroxypropylamino)anthraquinone.

The following compounds may be mentioned among the azine dyes: Basic Blue 17, Basic Red 2.

The following compounds may be mentioned among the triarylmethane dyes: Basic Green 1, Acid Blue 9, Basic Violet 3, Basic Violet 14, Basic Blue 7, Acid Violet 49, Basic Blue 26, Acid Blue 7.

The following compounds may be mentioned among the indoamine dyes: 2-β-hydroxyethylamino-5-[bis(β-4′-hydroxyethyl)amino]anilino-1,4-benzoquinone, 2-β-hydroxyethylamino-5-(2′-methoxy-4′-amino)anilino-1,4-benzoquinone, 3-N-(2′-chloro-4′-hydroxy)phenylacetylamino-6-methoxy-1,4-benzoquinone imine, 3-N-(3′-chloro-4′-methylamino)phenylureido-6-methyl-1,4-benzoquinone imine, 3-[4′-N-(ethyl,carbamylmethyl)amino]-phenylureido-6-methyl-1,4-benzoquinone imine.

Among the natural direct dyes which can be used according to the invention, there may be mentioned lawsone, juglone, alizarin, purpurin, carminic acid, kermesic acid, purpurogallin, protocatechaldehyde, indigo, isatin, curcumin, spinulosin, apigenidin. It is also possible to use extracts or decoctions containing these natural dyes and in particular poultices or extracts based on henna.

When the ready-to-use bleaching composition in accordance with the invention comprises one or more direct dyes, they are generally present in a quantity of between 0.001 and 20% by weight approximately of the total weight of the ready-to-use composition, and still more preferably between 0.005 and 10% by weight approximately.

The oxidation dye precursors may be chosen from the oxidation bases and couplers conventionally used in the dyeing field.

By way of examples of oxidation bases, there may be mentioned para-phenylenediamines, double bases, para-aminophenols, ortho-aminophenols, heterocyclic bases and addition salts thereof.

Among the para-phenylenediamines, there may be mentioned, by way of example, para-phenylenediamine, para-tolylenediamine, 2-chloro-para-phenylenediamine, 2, 3-dimethyl-para-phenylenediamine, 2,6-dimethyl-para-phenylenediamine, 2,6-diethyl-para-phenylenediamine, 2,5-dimethyl-para-phenylenediamine, N,N-dimethyl-para-phenylenediamine, N,N-diethyl-para-phenylenediamine, N,N-dipropyl-para-phenylenediamine, 4-amino-N,N-diethyl-3-methylaniline, N,N-bis(β-hydroxyethyl)-para-phenylenediamine, 4-N,N-bis(β-hydroxyethyl)amino-2-methylaniline, 4-N,N-bis(β-hydroxyethyl)amino-2-chloroaniline, 2-β-hydroxyethyl-para-phenylenediamine, 2-fluoro-para-phenylenediamine, 2-isopropyl-para-phenylenediamine, N-(β-hydroxypropyl)-para-phenylenediamine, 2-hydroxymethyl-para-phenylenediamine, N,N-dimethyl-3-methyl-para-phenylenediamine, N,N-(ethyl-β-hydroxyethyl)-para-phenylenediamine, N-(β,γ-dihydroxypropyl) -para-phenylenediamine, N-(4′-aminophenyl)-para-phenylenediamine, N-phenyl-para-phenylenediamine, 2-β-hydroxyethyloxy-para-phenylenediamine, 2-β-acetylaminoethyloxy-para-phenylenediamine, N-(β-methoxyethyl)-para-phenylenediamine, 4-amino-phenylpyrrolidine, 2-thienyl-para-phenylenediamine, 2-β-hydroxyethylamino-5-aminotoluene, 3-hydroxy-1-(4′-aminophenyl)pyrrolidine and their addition salts with an acid.

Among the para-phenylenediamines mentioned above, there are particularly preferred para-phenylenediamine, para-tolylenediamine, 2-isopropyl-para-phenylenediamine, 2-β-hydroxyethyl-para-phenylenediamine, 2-β-hydroxyethyloxy-para-phenylenediamine, 2,6-dimethyl-para-phenylenediamine, 2,6-diethyl-para-phenylenediamine, 2,3-dimethyl-para-phenylenediamine, N,N-bis(β-hydroxyethyl)-para-phenylenediamine, 2-chloro-para-phenylenediamine, 2-β-acetylaminoethyloxy-para-phenylenediamine, and their addition salts with an acid.

Among the double bases, there may be mentioned, by way of examples, bisphenylalkylenediamines and bis-para-aminophenols.

Among the bisphenylalkylenediamines, there may be mentioned, by way of example, N,N′-bis(β-hydroxyethyl)-N,N′-bis(4′-aminophenyl)-1,3-diaminopropanol, N,N′-bis(β-hydroxyethyl)-N,N′-bis(4′-aminophenyl)ethylenediamine, N,N′-bis(4-aminophenyl)tetramethylenediamine, N,N′-bis(β-hydroxyethyl)-N,N′-bis(4-aminophenyl)tetramethylenediamine, N,N′-bis(4-methylaminophenyl)tetramethylenediamine, N,N′-bis(ethyl)-N,N′-bis(4′-amino-3′-methylphenyl)ethylenediamine, 1,8-bis(2,5-diaminophenoxy)-3,6-dioxaoctane, and their addition salts with an acid.

Among the para-aminophenols, there may be mentioned, by way of example, para-aminophenol, 4-amino-3-methylphenol, 4-amino-3-fluorophenol, 4-amino-3-hydroxymethylphenol, 4-amino-2-methylphenol, 4-amino-2-hydroxymethylphenol, 4-amino-2-methoxymethylphenol, 4-amino-2-aminomethylphenol, 4-amino-2-(β-hydroxyethylaminomethyl)phenol, 4-amino-2-fluorophenol, and their addition salts with an acid.

Among the ortho-aminophenols, there may be mentioned, by way of example, 2-aminophenol, 2-amino-5-methylphenol, 2-amino-6-methylphenol, 5-acetamido-2-aminophenol, and their addition salts with an acid.

Among the heterocyclic bases, there may be mentioned, by way of example, pyridine derivatives, pyrimidine derivatives and pyrazole derivatives.

Among the pyridine derivatives, there may be mentioned the compounds described for example in Patents GB 1,026,978 and GE 1,153,196, such as 2,5-diaminopyridine, 2-(4-methoxyphenyl)amino-3-aminopyridine, 2,3-diamino-6-methoxypyridine, 2-(β-methoxyethyl)amino-3-amino-6-methoxypyridine, 3,4-diaminopyridine, and their addition salts with an acid.

Other pyridine oxidation bases useful in the present invention are the oxidation bases 3-aminopyrazolo[1,5-a]pyridines or their addition salts which are described, for example, in Patent Application FR 2801308. By way of example, there may be mentioned pyrazolo[1,5-a]pyridin-3-ylamine; 2-acetylamino-pyrazolo[1,5-a]pyridin-3-ylamine; 2-morpholin-4-ylpyrazolo[1,5-a]pyridin-3-ylamine; 3-aminopyrazolo[1,5-a]pyridine-2-carboxylic acid; 2-methoxypyrazolo[1,5-a]pyridin-3-ylamino; (3-aminopyrazolo[1,5-a]pyridin-7-yl)methanol; 2-(3-aminopyrazolo[1,5-a]pyridin-5-yl)ethanol; 2-(3-aminopyrazolo[1,5-a]pyridin-7-yl)ethanol; (3-aminopyrazolo[1,5-a]pyridin-2-yl)methanol; 3,6-diaminopyrazolo[1,5-a]pyridine; 3,4-diaminopyrazolo[1,5-a]pyridine; pyrazolo[1,5-a]pyridine-3,7-diamine; 7-morpholin-4-ylpyrazolo[1,5-a]pyridin-3-ylamine; pyrazolo[1,5-a]pyridine-3,5-diamine; 5-morpholin-4-ylpyrazolo[1,5-a]pyridin-3-ylamine; 2-[(3-aminopyrazolo[1,5-a]pyridin-5-yl)-(2-hydroxyethyl)amino]ethanol; 2-[(3-aminopyrazolo[1,5-a]pyridin-7-yl) (2-hydroxyethyl)amino]ethanol; 3-aminopyrazolo[1,5-a]pyridin-5-ol; 3-aminopyrazolo[1,5-a]pyridin-4-ol, 3-aminopyrazolo[1,5-a]pyridin-6-ol; 3-aminopyrazolo[1,5-a]pyridin-7-ol; and their addition salts with an acid or with a base.

Among the pyrimidine derivatives, there may be mentioned the compounds described for example in Patents DE 2,359,399; JP 88-169,571; JP 05-63124; EP 0770375 or Patent Application WO 96/15765, such as 2,4,5,6-tetraaminopyrimidine, 4-hydroxy-2,5,6-triaminopyrimidine, 2-hydroxy-4,5,6-triaminopyrimidine, 2,4-dihydroxy-5,6-diaminopyrimidine, 2,5,6-triaminopyrimidine, and the pyrazolopyrimidine derivatives such as those mentioned in Patent Application FR-A-2,750,048 and among which there may be mentioned pyrazolo[1,5-a]pyrimidine-3,7-diamine; 2,5-dimethylpyrazolo[1,5-a]pyrimidine-3,7-diamine; pyrazolo[1,5-a]pyrimidine-3,5-diamine; 2,7-dimethylpyrazolo[1,5-a]pyrimidine-3,5-diamine; 3-aminopyrazolo[1,5-a]pyrimidin-7-ol; 3-aminopyrazolo[1,5-a]pyrimidin-5-ol; 2-(3-aminopyrazolo[1,5-a]pyrimidin-7-ylamino)ethanol, 2-(7-aminopyrazolo[1,5-a]pyrimidin-3-ylamino)ethanol, 2-[(3-aminopyrazolo[1,5-a]pyrimidin-7-yl) (2-hydroxyethyl)amino]ethanol, 2-[(7-aminopyrazolo[1,5-a]-pyrimidin-3-yl) (2-hydroxyethyl)amino]ethanol, 5,6-dimethylpyrazolo[1,5-a]pyrimidine-3,7-diamine, 2,6-dimethylpyrazolo[1,5-a]pyrimidine-3,7-diamine, 2,5,N7,N7-tetramethylpyrazolo[1,5-a]pyrimidine-3,7-diamine, 3-amino-5-methyl-7-imidazolylpropylaminopyrazolo[1,5-a]pyrimidine and their addition salts with an acid and their tautomeric forms, when a tautomeric equilibrium exists.

Among the pyrazole derivatives, there may be mentioned the compounds described in Patents DE 3,843,892, DE 4,133,957 and Patent Applications WO 94/08969, WO 94/08970, FR-A-2,733,749 and DE 195 43 988 such as 4,5-diamino-1-methylpyrazole, 4,5-diamino-1-(β-hydroxyethyl)pyrazole, 3,4-diaminopyrazole, 4,5-diamino-1-(4′-chlorobenzyl)pyrazole, 4,5-diamino-1,3-dimethylpyrazole, 4,5-diamino-3-methyl-1-phenylpyrazole, 4,5-diamino-1-methyl-3-phenylpyrazole, 4-amino-1,3-dimethyl-5-hydrazinopyrazole, 1-benzyl-4,5-diamino-3-methylpyrazole, 4,5-diamino-3-tert-butyl-1-methylpyrazole, 4,5-diamino-1-tert-butyl-3-methylpyrazole, 4,5-diamino-1-(β-hydroxyethyl)-3-methylpyrazole, 4,5-diamino-1-ethyl-3-methylpyrazole, 4,5-diamino-1-ethyl-3-(4′-methoxyphenyl)pyrazole, 4,5-diamino-1-ethyl-3-hydroxymethylpyrazole, 4,5-diamino-3-hydroxymethyl-1-methylpyrazole, 4,5-diamino-3-hydroxymethyl-1-isopropylpyrazole, 4,5-diamino-3-methyl-1-isopropylpyrazole, 4-amino-5-(2′-aminoethyl)amino-1,3-dimethylpyrazole, 3,4,5-triaminopyrazole, 1-methyl-3,4,5-triaminopyrazole, 3,5-diamino-1-methyl-4-methylaminopyrazole, 3,5-diamino-4-(β-hydroxyethyl)amino-1-methylpyrazole, and their addition salts with an acid.

When the ready-to-use bleaching composition in accordance with the invention comprises one or more oxidation bases, they are generally present in a quantity of between 0.001 and 10% by weight approximately of the total weight of the ready-to-use composition, preferably between 0.005 and 6% by weight approximately.

By way of examples of couplers, there may be mentioned meta-phenylenediamines, meta-aminophenols, meta-diphenols, naphthalene couplers, heterocyclic couplers and addition salts thereof.

By way of examples, there may be mentioned 2-methyl-5-aminophenol, 5-N-(β-hydroxyethyl)amino-2-methylphenol, 6-chloro-2-methyl-5-aminophenol, 3-aminophenol, 1,3-dihydroxybenzene, 1,3-dihydroxy-2-methylbenzene, 4-chloro-1,3-dihydroxybenzene, 2,4-diamino-1-(β-hydroxyethyloxy)benzene, 2-amino-4-(β-hydroxyethylamino)-1-methoxybenzene, 1,3-diaminobenzene, 1,3-bis(2,4-diaminophenoxy)propane, 3-ureidoaniline, 3-ureido-1-dimethylaminobenzene, sesamol, 1-β-hydroxyethylamino-3,4-methylenedioxybenzene, α-naphthol, 2-methyl-1-naphthol, 6-hydroxyindole, 4-hydroxyindole, 4-hydroxy-N-methylindole, 2-amino-3-hydroxypyridine, 6-hydroxybenzomorpholine, 3,5-diamino-2,6-dimethoxypyridine, 1-N-(β-hydroxyethyl)amino-3,4-methylenedioxybenzene, 2,6-bis(β-hydroxyethylamino)toluene and their addition salts with an acid.

When the ready-to-use bleaching composition in accordance with the invention comprises one or more couplers, they are generally present in a quantity of between 0.001 and 10% by weight approximately of the total weight of the ready-to-use composition, preferably between 0.005 and 6% by weight approximately.

In general, the addition salts of the oxidation bases and couplers which can be used in the context of the invention are in particular chosen from the addition salts with an acid such as the hydrochlorides, hydrobromides, sulphates, citrates, succinates, tartrates, lactates, tosylates, benzenesulphonates, phosphates and acetates and the addition salts with a base such as sodium hydroxide, potassium hydroxide, ammonium hydroxide, amines or alkanolamines.

According to a particular embodiment, the ready-to-use bleaching composition in accordance with the invention comprises at least one alkaline agent as defined above. It may then result from mixing the invert emulsion in accordance with the invention with an aqueous composition comprising the alkaline agent(s) in an appropriate cosmetic medium.

According to another particular embodiment, the ready-to-use bleaching composition in accordance with the invention comprises at least one alkaline agent and at least one persalt as defined above. It may then result from mixing the invert emulsion in accordance with the invention with an anhydrous composition comprising the alkaline agent(s) and the persalt(s) in an appropriate cosmetic medium. It may also result from mixing the invert emulsion in accordance with the invention with an aqueous composition comprising the alkaline agent(s) in an appropriate cosmetic medium and an anhydrous composition comprising the persalt(s) in an appropriate cosmetic medium.

According to another particular embodiment, the ready-to-use bleaching composition in accordance with the invention comprises at least one direct dye as defined above. It may then result from mixing the invert emulsion in accordance with the invention with an aqueous composition comprising the direct dye(s) in an appropriate cosmetic medium.

According to another particular embodiment, the ready-to-use bleaching composition in accordance with the invention comprises at least one alkaline agent and at least one direct dye and/or at least one oxidation dye precursor as defined above. It may then result from mixing the invert emulsion in accordance with the invention with an aqueous composition comprising, in an appropriate cosmetic medium, the alkaline agent(s) and the direct dye(s) and/or the oxidation dye precursor(s). It may also result from mixing the invert emulsion in accordance with the invention with an aqueous composition comprising the alkaline agent(s) in an appropriate cosmetic medium and an aqueous composition comprising the direct dye(s) and/or the oxidation dye precursor(s) in an appropriate cosmetic medium.

According to another particular embodiment, the ready-to-use bleaching composition in accordance with the invention comprises at least one alkaline agent, at least one persalt and at least one direct dye as defined above. It may then result from mixing the invert emulsion in accordance with the invention with an anhydrous composition comprising the alkaline agent(s) and the persalt(s) in an appropriate cosmetic medium and an aqueous composition comprising the direct dye(s) in an appropriate cosmetic medium. It may also result from mixing the invert emulsion in accordance with the invention with an aqueous composition comprising the alkaline agent(s) in an appropriate cosmetic medium, an anhydrous composition comprising the persalt(s) in an appropriate cosmetic medium and an aqueous composition comprising the direct dye(s) in an appropriate cosmetic medium.

The compositions comprising the persalt(s) are anhydrous. They may also comprise customary additives in the field, in particular water-soluble thickening polymers, fillers such as clays or amorphous silica, binders such as vinylpyrrolidone, lubricants such as polyol stearates or alkali or alkaline-earth metal stearates, and agents for controlling the emission of oxygen such as magnesium carbonate or oxide, dyeing agents or mattifying agents such as titanium oxides or anionic, nonionic, cationic or amphoteric surfactants, vitamins.

By way of illustration, the content of additive(s) represents from 0.01 to 40% by weight, preferably from 0.1 to 30% by weight relative to the total weight of the compositions.

The anhydrous compositions may be provided in powdered or paste form. In the case where they are provided in paste form, they further comprise an organic inert liquid chosen from polydecenes of formula C₁₀ₙH_{[(20n)+2]} in which n varies from 3 to 9 and preferably from 3 to 7, fatty alcohol or fatty acid esters, C₁₂-C₂₄ fatty acid esters or diesters of sugars, cyclic ethers or cyclic esters, silicone oils, mineral oils or vegetable oils.

The other compositions are preferably aqueous. The appropriate cosmetic medium for these compositions generally consists of water or of a mixture of water and at least one organic solvent for solubilizing the compounds which might not be sufficiently soluble in water. By way of organic solvent, there may for example be mentioned C₁-C₄ lower alkanols such as ethanol and isopropanol; glycerol; glycols and glycol ethers such as 2-butoxyethanol, propylene glycol, propylene glycol monomethyl ether, and aromatic alcohols such as benzyl alcohol or phenoxyethanol, analogous products and mixtures thereof.

The solvents may be present in proportions preferably between 1 and 40% by weight relative to the total weight of the composition, and more preferably still between 5 and 30% by weight approximately.

These compositions may also contain various adjuvants conventionally used in compositions for bleaching hair, such as anionic, cationic, nonionic, amphoteric or zwitterionic surfactants or mixtures thereof, anionic, cationic, nonionic, amphoteric or zwitterionic polymers or mixtures thereof, inorganic or organic thickeners, and in particular the associative thickeners, anionic, cationic, nonionic and amphoteric polymers, antioxidants, penetrating agents, sequestrants, perfumes, buffers, dispersing agents, conditioning agents such as, for example, modified or unmodified, volatile or nonvolatile silicones, film-forming agents, ceramides, preservatives, opacifying agents.

The above adjuvants are generally present in a quantity, for each of them, of between 0.01 and 20% by weight relative to the weight of the composition.

Of course, persons skilled in the art will be careful to choose this or these optional additional compounds such that the advantageous properties intrinsically attached to these compositions are not, or not substantially, impaired by the addition(s) envisaged.

These compositions may be provided in various forms, such as in the form of liquids, creams, gels, or in any other appropriate cosmetic form.

The pH of the ready-to-use bleaching composition in accordance with the invention is between 4 and 11. It is preferably alkaline, and more preferably still between 7 and 11.

The bleaching method in accordance with the present invention consists in applying to the keratin fibres an invert emulsion or a ready-to-use bleaching composition in accordance with the invention as defined above.

The subject of the present invention is also a kit for bleaching keratin fibres, containing an invert emulsion in accordance with the invention and one or more compositions in which at least one alkaline agent and/or at least one persalt and/or at least one dye precursor and/or at least one direct dye as defined above are distributed.

According to a particular embodiment of the invention, the kit in accordance with the invention contains an invert emulsion in accordance with the invention and an aqueous composition comprising at least one alkaline agent as defined above in an appropriate cosmetic medium.

According to another particular embodiment of the invention, the kit in accordance with the invention contains an invert emulsion in accordance with the invention, an anhydrous composition comprising at least one persalt as defined above in an appropriate cosmetic medium and an aqueous composition comprising at least one alkaline agent as defined above in an appropriate cosmetic medium.

According to another particular embodiment of the invention, the kit in accordance with the invention contains an invert emulsion in accordance with the invention and an anhydrous composition comprising at least one alkaline agent and at least one persalt as defined above in an appropriate cosmetic medium.

According to another particular embodiment of the invention, the kit in accordance with the invention contains an invert emulsion in accordance with the invention and an aqueous composition comprising at least one direct dye as defined above in an appropriate cosmetic medium.

According to another particular embodiment of the invention, the kit in accordance with the invention contains an invert emulsion in accordance with the invention, an aqueous composition comprising at least one alkaline agent as defined above in an appropriate cosmetic medium and an aqueous composition comprising at least one direct dye and/or at least one oxidation dye precursor as defined above in an appropriate cosmetic medium.

According to another particular embodiment of the invention, the kit in accordance with the invention contains an invert emulsion in accordance with the invention and an aqueous composition comprising, in an appropriate cosmetic medium, at least one alkaline agent and at least one direct dye and/or at least one oxidation dye precursor as defined above.

According to another particular embodiment of the invention, the kit in accordance with the invention contains an invert emulsion in accordance with the invention, an anhydrous composition comprising at least one alkaline agent and at least one persalt as defined above in an appropriate cosmetic medium and an aqueous composition comprising at least one direct dye as defined above in an appropriate cosmetic medium.

According to another particular embodiment of the invention, the kit in accordance with the invention contains an invert emulsion in accordance with the invention, an anhydrous composition comprising at least one persalt as defined above in an appropriate cosmetic medium, an aqueous composition comprising at least one alkaline agent as defined above in an appropriate cosmetic medium and an aqueous composition comprising at least one direct dye as defined above in an appropriate cosmetic medium.

The subject of the present invention is also the use, for bleaching keratin fibres, of an invert emulsion or of a ready-to-use bleaching composition in accordance with the invention as defined above.

The following examples serve to illustrate the invention without however being limiting in nature.

### EXAMPLE

The following oxidizing composition 1 was prepared:

OXIDIZING COMPOSITION 1

| | | |
|---|---|---|
| Paraffin oil | 40 | g |
| Arlacel P135 | 4 | g |
| Arlamol ISML | 1 | g |
| Ammonium | 2 | g |
| acryloyldimethyltaurate/steareth- | | |
| 25 methacrylate crosspolymer | | |
| Magnesium sulphate | 0.3 | g |
| Hydrogen peroxide | 6 | g |
| Sodium stannate•6 H₂O | 0.04 | g |
| Diethylenetriaminepentaacetic | 0.015 | g |
| acid | | |
| Tetrasodium pyrophosphate•10 H₂O | 0.03 | g |

| | |
|---|---|
| Phosphoric acid | qs pH = 3 |
| Demineralized water | qs 100 g |

The oxidizing composition 1 is mixed at the time of use with a bleaching powder containing 50% of persulphates, 24.1% of silicates and 2.6% of chloride of ammonium, with a bleaching powder/oxidizing composition ratio equal to 1/1.5. Mixture A is obtained.

By way of reference, an oxidizing composition containing 6% of hydrogen peroxide and having a pH equal to 2 is mixed at the time of use with the same bleaching powder with a bleaching powder/oxidizing composition ratio equal to 1/1.5. Mixture B is obtained.

The mixtures obtained are applied to 2.5 g of locks of natural chestnut brown hair in an amount of 10 g of mixture per 1 g of hair. After a leave-in time of 40 minutes, the locks are rinsed, washed with a standard shampoo, rinsed again and then dried.

The results are described in the table below.

COMPARISON OF THE BLEACHING LEVELS

| | Mixture A | Mixture B |
|---|---|---|
| | (invention) | (prior art) |
| Bleaching effect | TH = 9.25 | TH = 8.5 |
| Tone height of the | | |
| initial natural chest- | | |
| nut brown hair colour: | | |
| TH = 4 | | |

It is observed that the oxidizing composition in accordance with the invention makes it possible to obtain a higher degree of lightening than the known prior art oxidizing composition.

1-27. (canceled)

28. An invert emulsion for bleaching keratin fibers, comprising:
an inert phase comprising at least one nonoxygenated and nonperfluorinated liquid compound having a water-solubility at 25° C. of less than 1%; and
an aqueous hydrogen peroxide solution;
wherein the inert phase is present in a concentration of at least 20% by weight of the total weight of the invert emulsion; and
further wherein the mean diameter by volume of the dispersed aqueous phase is greater than 200 nm.

29. The invert emulsion according to claim 28, wherein the at least one nonoxygenated and nonperfluorinated liquid compound is chosen from polyalphaolefins, polybutenes and polyisobutenes, mineral oils, paraffin oils, and polystyrene and polyisoprene block copolymers.

30. The invert emulsion according to claim 28, wherein the inert phase is present in a concentration ranging from 20% to 95% by weight of the total weight of the invert emulsion.

31. The invert emulsion according to claim 30, wherein the inert phase is present in a concentration ranging from 30% to 90% by weight of the total weight of the invert emulsion.

32. The invert emulsion according to claim 28, wherein the mean diameter by volume of the dispersed aqueous phase ranges from 200 nm to 100 μm.

33. The invert emulsion according to claim 28, wherein the concentration of the aqueous hydrogen peroxide solution ranges from 1% to 20% by weight of the total weight of the invert emulsion.

34. The invert emulsion according to claim 33, wherein the concentration of the aqueous hydrogen peroxide solution ranges from 2% to 12% by weight of the total weight of the invert emulsion.

35. The invert emulsion according to claim 28, further comprising at least one compound chosen from surfactants, derivatives of polyisobutylene succinic anhydrides, amphiphilic polymers, and thickening polymers.

36. The invert emulsion according to claim 28, further comprising at least one surfactant chosen from anionic surfactants having at least one chain chosen from linear C₆-C₂₂ alkylated chains, branched C₆-C₂₂ alkylated chains, and perfluorinated chains, nonionic surfactants having at least one chain chosen from linear C₆-C₂₂ alkylated chains, branched C₆-C₂₂ alkylated chains, and perfluorinated chains, cationic surfactants having at least one chain chosen from linear C₆-C₂₂ alkylated chains, branched C₆-C₂₂ alkylated chains, and perfluorinated chains, and amphoteric surfactants having at least one chain chosen from linear C₆-C₂₂ alkylated chains, branched C₆-C₂₂ alkylated chains, and perfluorinated chains.

37. The invert emulsion according to claim 28, further comprising at least one surfactant chosen from silicone surfactants.

38. The invert emulsion according to claim 28, further comprising at least one surfactant, wherein said at least one surfactant is each present in a concentration ranging from 0.1% to 30% by weight of the total weight of the invert emulsion.

39. The invert emulsion according to claim 28, further comprising at least one derivative of polyisobutylene succinic anhydrides chosen from anionic derivatives of polyisobutylene succinic anhydrides, nonionic derivatives of polyisobutylene succinic anhydrides, amphoteric derivatives of polyisobutylene succinic anhydrides, and cationic derivatives of polyisobutylene succinic anhydrides.

40. The invert emulsion according to claim 28, further comprising at least one amphiphilic polymer chosen from water-soluble or water-dispersible amphiphilic block copolymers, oxyethylenated or nonoxyethylenated, telechelic or graft associative polymers bearing on their main chain at least one group chosen from C₆-C₂₂ alkyl groups and C₆-C₂₂ aryl groups,
wherein said at least one amphiphilic polymer may be chosen from amphiphilic polymers having at least one charge chosen from cationic charges, anionic charges, amphoteric charges, and nonionic charges.

41. The invert emulsion according to claim 28, further comprising at least one thickening polymer chosen from crosslinked acrylic polymers, natural polysaccharides, and modified polysaccharides.

42. The invert emulsion according to claim 28, further comprising at least one compound chosen from derivatives of polyisobutylene succinic anhydrides, amphiphilic polymers, and thickening polymers, and
wherein said at least one compound is each present in a concentration ranging from 0.1% to 30% by weight of the total weight of the invert emulsion.

43. The invert emulsion according to claim 28, wherein the pH of the dispersed aqueous phase of said invert emulsion is less than 5.

44. A ready-to-use bleaching composition resulting from mixing an invert emulsion according to claim 28 with at least one composition comprising at least one compound chosen from alkaline agents, persalts, direct dyes, and oxidation dye precursors.

45. The ready-to-use bleaching composition according to claim 44, wherein the alkaline agents are chosen from organic amines, ammonia, and silicates.

46. The ready-to-use bleaching composition according to claim 44, wherein the alkaline agents are present in a concentration ranging from 0.01% to 40% by weight of the total weight of the ready-to-use composition.

47. The ready-to-use bleaching composition according to claim 44, wherein the at least one persalt is chosen from ammonium perborates, ammonium percarbonates and ammonium persulphates, alkali metal perborates, alkali metal percarbonates, and alkali metal persulphates.

48. The ready-to-use bleaching composition according to claims 44, wherein the at least one persalt is present in a concentration ranging from 10% to 70% by weight of the total weight of the ready-to-use composition.

49. The ready-to-use bleaching composition according to claim 44, wherein the at least one direct dye is present in a concentration ranging from 0.001% to 20% by weight of the total weight of the ready-to-use composition.

50. The ready-to-use bleaching composition according to claim 44, wherein the at least one dye precursor is chosen from oxidation bases and couplers.

51. The ready-to-use bleaching composition according to claim 44, wherein the at least one dye precursor is present in a concentration ranging from 0.001% to 10% by weight of the total weight of the ready-to-use composition.

52. The method for bleaching keratin fibers, wherein an invert emulsion according to claim 28 or a ready-to-use bleaching composition according to claim 44 is applied to the keratin fibers.

53. A kit for bleaching keratin fibers, comprising an invert emulsion according to claim 28 and at least one composition comprising at least one compound chosen from alkaline agents, persalts, dye precursors, and direct dyes.

54. The kit for bleaching keratin fibers according to claim 53, wherein:
the alkaline agents are chosen from organic amines, ammonia and silicates;
the persalts are chosen from ammonium perborates, ammonium percarbonates, ammonium persulphates, alkali metal perborates, alkali metal percarbonates, and alkali metal persulphates; and
the dye precursors are chosen from oxidation bases and couplers.
